(19) United States
(12) Patent Application Publication
(10) Pub. No.: US 2013/0302243 A1
(43) Pub. Date: Nov. 14, 2013

(54) RADIOLABELED NANOSYSTEM, PROCESS FOR THE PREPARATION THEREOF AND ITS USE

(71) Applicant: BBS NANOMEDICINA ZRT., Debrecen (HU)

(72) Inventors: János Borbély, Debrecen (HU); Magdolna Bodnár, Debrecen (HU); Lajos Balogh, Budapest (HU); András Polyák, Budapest (HU)

(73) Assignee: BBS NanoMedicina Zrt., Debrecen (HU)

(21) Appl. No.: 13/889,198

(22) Filed: May 7, 2013

Related U.S. Application Data
(60) Provisional application No. 61/644,611, filed on May 9, 2012.

(51) Int. Cl. A61K 51/04; A61K 51/12; A61K 51/06

(52) U.S. Cl. CPC A61K 51/048; A61K 51/065; A61K 51/1244
USPC 424/1.37

(57) ABSTRACT

Disclosed are novel, targeted, self-assembled nanoparticles radiolabeled with technetium-99m (Tc-99m) as radiodiagnostic compositions, methods of using these compositions and methods for preparing such radiolabeled compositions. Specifically, the compositions of the nanoparticles are composed of self-assembled polyelectrolyte biopolymers having targeting moieties, which can be suitable for targeted delivery of radionuclide metal ions complexed to the nanoparticles. These radiolabeled nanoparticles can specifically bind and internalize into the targeted tumor cells to realize the receptor mediated uptake. Radiolabeled, targeted nanoparticulate composition, methods for making, radiolabeling and using such compositions in the field of diagnosis and therapy are also provided.

### CROSS-REFERENCE TO RELATED APPLICATION

This application takes the priority of U.S. Provisional Patent Application Ser. No. 61/644,611, filed on the 9 May 2012, the entire content of which is incorporated herein by reference.

### BRIEF DESCRIPTION OF THE INVENTION

The present invention discloses novel, targeted, self-assembled nanoparticles radiolabeled with technetium-99m (Tc-99m) as radiodiagnostic compositions, methods of using these compositions and methods for preparing such radiolabeled compositions. Specifically, the compositions of the nanoparticles are composed of self-assembled polyelectrolyte biopolymers having targeting moieties, which can be suitable for targeted delivery of radionuclide metal ions complexed to the nanoparticles. These radiolabeled nanoparticles can specifically bind and internalize into the targeted tumor cells to realize the receptor mediated uptake. Radiolabeled, targeted nanoparticulate composition, methods for making, radiolabeling and using such compositions in the field of diagnosis and therapy are also provided.

### REFERENCES CITED

PATENT DOCUMENTS

| | | |
|---|---|---|
| U.S. Pat. No. 4,444,744 | April 1984 | Goldberg |
| U.S. Pat. No. 4,606,907 | August 1986 | Simon et al. |
| U.S. Pat. No. 4,765,971 | August 1988 | Wester et al. |
| U.S. Pat. No. 4,915,931 | April 1990 | Yokoyama et al. |
| U.S. Pat. No. 4,916,214 | April 1990 | Chiu et al. |
| U.S. Pat. No. 5,059,541 | October 1991 | Fritzberg et al. |
| U.S. Pat. No. 5,071,965 | December 1991 | Dunn et al. |
| U.S. Pat. No. 5,328,679 | July 1994 | Hansen et al. |
| U.S. Pat. No. 5,330,738 | July 1994 | Nosco |
| U.S. Pat. No. 5,552,525 | September 1996 | Dean |
| U.S. Pat. No. 5,866,544 | February 1999 | Goodbody et al. |
| U.S. Pat. No. 6,066,310 | May 2000 | Konishi et al. |
| WO 2010/087959 A1 | January 2010 | Magneson et al. |
| WO 2006/080993 | August 2006 | Liu et al. |
| WO 2004/037297 | May 2004 | Brauers et al. |

### OTHER PUBLICATIONS

Wenyan Guo, Huihui Jing, Wenjiang Yang, Zhide Guo, Shi Feng, Xianzhong Zhang, Radiolabeling of folic acid-modified chitosan with ^{99m}Tc as potential agents for folate-receptor-mediated targeting, Bioorganic and Medicinal Chemistry Letters 21 (2011) 6446-6450.
Misara Hamoudeh, Muhammad Anas Kamleh, Roudayna Diab, Hatem Fessi, Radionuclides delivery systems for nuclear imaging and radiotherapy of cancer, Advanced Drug Delivery Reviews 60 (2008) 1329-1346.
Ripen Misri, Dominik Meier, Andrew C. Yung, Piotr Kozlowski, Urs O. Häfeli, Development and evaluation of a dual-modality (MRI/SPECT) molecular imaging bioprobe, Nanomedicine: Nanotechnology, Biology, and Medicine, doi: 10.1016/j.nano.2011.10.013
George Loudos, George C. Kagadis, Dimitris Psimadas, Current status and future perspectives of in vivo small animal imaging using radiolabeled nanoparticles, European Journal of Radiology 78 (2011) 287-295.
O. C. Boerman, P. Layerman, W. J. G. Oyen, F. H. M. Corstens, G. Storm, Radiolabeled liposomes for scintigraphic imaging, Progress in Lipid Research 39 (2000) 461-475.
Misara Hamoudeh, Muhammad Anas Kamleh, Roudayna Diab, Hatem Fessi, Radionuclides delivery systems for nuclear imaging and radiotherapy of cancer, Advanced Drug Delivery Reviews 60 (2008) 1329-1346.
Chun-Yen Ke, Carla J. Mathias, Mark A. Green, The folate receptor as a molecular target for tumor-selective radionuclide delivery, Nuclear Medicine and Biology 30 (2003) 811-817.
Chun-Yen Ke, Carla J. Mathias, Mark A. Green, Folate-receptor-targeted radionuclide imaging agents, Advanced Drug Delivery Reviews 56 (2004) 1143-1160.
Wonjung Kwak, Hee-Seong Jang, Takele Belay, Jinu Kim, Yeong Su Ha, Sang Woo Lee, Byeong-Cheol Ahn, Jaetae Lee, Kwon Moo Park, Jeongsoo Yoo, Evaluation of kidney repair capacity using 99mTc-DMSA in ischemia/reperfusion injury models, Biochemical and Biophysical Research Communications 406 (2011) 7-12.

### FIELD OF THE INVENTION

The present invention relates to targeted, self-assembled nanoparticulate compositions that are radiodiagnostic imaging agents, methods of using these compositions and methods for preparing such radiolabeled compositions. Specifically, the invention relates to targeted nanoparticles labeled with gamma radiation-emitting radioisotopes such as technetium-99m (Tc-99m) useful as radioactive diagnostic imaging agents for SPECT (Single Photon Emission Computed Tomography). The novel targeted, radiolabeled nanoparticles as radioactive diagnostic imaging agent and methods for their production and use are also disclosed.

### BACKGROUND OF THE INVENTION

In the field of nuclear medicine, radionuclide metal ions are widely used for therapeutic and diagnostic applications. Gamma-emitting radionuclide metal ions, such as technetium-99m (Tc-99m) are ideal radioisotope for use in nuclear medicine. Tc-99m has an appropriate half-life (about 6 hours) and emits energy of gamma rays (about 140 KeV) with no alpha and beta radiation. Additionally, Tc-99m is easily prepared and available at low cost due to the result of development of Tc-99m generator.

Technetium-99m is obtained from generators as pertechnetate ion (TcO₄⁻) in the +7 oxidation state. In favor of formation of any complexes, Tc must be in lower atomic state (i.e. +3, +4, +5), which could be obtained by reducing agent. The most often used reducing agent is Sn²⁺ in the presence of a complexing agent. This reaction takes usually place in aqueous saline solution, preferably physiological saline solution suitable for intravenous injection.

Many recent attempts have been made to create radiolabeled diagnostic and pharmaceutical agent for application as sensitive imaging agent.

Recently, both small molecular and macromolecular radiopharmaceuticals have attracted much interest because of their ability to improve diagnosis. However, low-molecular weight radiopharmaceuticals are non-specific extracellular imaging agents and can have serious shortcomings, such as a short half-life in the blood, rapid diffusion out of the blood and excretion through the kidney, resulting in low image quality and a lack of targeting specificity.

In an effort to overcome these shortcomings, several macromolecular imaging agents have been developed for biomedical applications, including proteins, polysaccharides, liposomes, dendritic nanodevices, other natural and synthetic biocompatible polymers and polyelectrolyte complexes.

Macromolecular imaging agents have several advantages. These radiopharmaceuticals easily penetrate cellular membranes through active and passive mechanisms due to their small size, allowing them to act as drug carriers with tunable pharmacokinetic properties, enabling slow or sustained release of their payload.

Ideally, due to their colloid size, they circulate in the blood for sufficiently long time and targets the specific studied (cancer) cells to produce high relative activity intensity and to allow completion of the imaging procedure; afterwards, it should be degraded and excreted through the kidneys. In addition, these nano-sized colloid systems can be modified flexibly via their functional groups, and multiple, targeted nanocarriers can be formed.

Hydrophilic polymer macromolecules can behave as a polyelectrolyte due to their charged functional groups in aqueous media. Based on the attractive interaction of oppositely charged functional groups of polyelectrolytes they can self-assembly and can result in stable polyelectrolyte complexes. The polyelectrolyte complexes dispose several advantages, such as numerous reactive functional groups, the flexibility of the system and a lack of new covalent bond, which could modify the favorable biological properties of biopolymers.

The self-assembly of polyelectrolytes produces stable polyelectrolyte complexes, which can appear in nanoparticles, nanosystems, films or hydrogels. A variety of studies have focused on the preparation and characterization of these polyelectrolyte complexes, because these systems open many new opportunities to develop delivery of bioactive molecules. Several polyelectrolyte complex systems were developed for use as carrier for drug or gene delivery. After self-assembly, the residual functional groups of polyelectrolytes are available for transport and for targeting of active agents.

Targeting radiopharmaceuticals, as imaging agents internalize and accumulate selectively in the targeted specific cells, tissues, therefore a smaller dose is sufficient to realize high relative activity intensity between the examined and surrounded tissue areas. These radiolabeled systems contain active targeting molecule, which enables the specific binding and receptor mediated uptake of contrast agent into the targeted tumor cells.

Ideally, polymer-based radiopharmaceuticals, as imaging agents internalize and accumulate in the targeted tumor cell. Small doses of targeted radiopharmaceuticals are sufficient to produce high relative activity intensity (e.g. in SPECT) and to allow completion of the imaging procedure; afterwards it should be degraded and excreted through the kidneys. These systems contain active targeting moiety, which enables the specific binding and internalization of radiopharmaceuticals into the targeted tumor cells.

Targeting ligands include small molecules (e.g. folic acid), peptides (e.g. LHRH), monoclonal antibodies (e.g. Transtuzumab) or others.

Folic acid is a widely used targeting moiety of carrier for cancer therapy. It has been shown, that several human tumor cells overexpress folate receptors, and possess a high affinity for folic acid molecules. However normal tissues possess restricted number of folate receptors. Folate receptor is a valuable molecular target for tumor selective radionuclide delivery and therapy that is in approximately 90% highly expressed on a variety of cancers as ovarian carcinomas, endometrial, kidney, lung, breast, brain cancers or mesothelioma and myeloid leukemia.

Chitosan (CH) is a renewable, basic linear polysaccharide, containing β-[1→4]-linked 2-acetamido-2-deoxy-D-glucopyranose and 2-amino-2-deoxy-D-glucopyranose units with reactive amino groups. Because of its special set of properties, which include low or non-toxicity, biocompatibility, biodegradability, low or no immunogenicity and antibacterial properties, chitosan has found wide application in a variety of areas, such as biomedicine, pharmaceuticals, metal chelation, food additives, and other industrial applications. Its application could be difficult because of its low solubility in aqueous media. Chitosan can be solubilized by the protonation of its amino groups in acidic media, resulting in a cationic polysaccharide with high charge density appearing in viscous solution.

Poly-γ-glutamic acid (γ-PGA) consists of repetitive glutamic acid units connected by amide linkages between α-amino and γ-carboxylic acid functional groups. Γ-PGA is different from other proteins, in that glutamate is polymerized via the non-peptide γ-amide linkages, and thus is synthesized in a ribosome-independent manner. In could be prepared by bacterial fermentation with molecular weight range between 10 kDa and 1000 kDa.

γ-PGA is a hydrophilic, water soluble, biodegradable, edible and nontoxic polypeptide. It is a polyanion having reactive carboxyl groups; it is non-toxic for the environment and humans. Therefore, γ-PGA and its derivatives have been employed extensively in a variety of commercial applications such as cosmetics, food, medicine, and water treatment.

### BACKGROUND OF THE PRIOR ART

Several US patents describe complexes, complex compounds as radiopharmaceutical imaging agents. Yokoyama et al. (U.S. Pat. No. 4,915,931) describe Tc-99m mononuclide complex compound; Simón et al. (U.S. Pat. No. 4,606,907) relate to bone seeking Tc-99m complexes; Dunn et al (U.S. Pat. No. 5,071,965) provide complexes comprising Tc-99m in the +4 oxidation state bonded to a special ligand, where the formula of the ligand is claimed; Nosco (U.S. Pat. No. 5,330,738) describes radiopharmaceutical compositions comprising Tc-99m complexes with special ligand structures; Brauers et al. (WO 2004/037297) relate to aza-diaminedioxime conjugate technetium-99m metal complex compositions.

Cationic complexes of Tc-99m are also described in numerous patents: e.g. Wester et al. (U.S. Pat. No. 4,765,971) describe a special Tc-99m-arene complex; Chiu et al. (U.S. Pat. No. 4,916,214) provide cationic complexes of Tc-99m with special ligand structures, Liu et al. (WO 2006/080993) describe novel cationic crown-ether containing metal complexes with special structures and radiopharmaceutical compositions comprising these cationic crown-ether containing radionuclide metal ion complexes.

Goldberg (U.S. Pat. No. 4,444,744) relates tumor localization and therapy with labeled antibodies.

Fritzberg et al. (U.S. Pat. No. 5,059,541) relate to minimal derivatization of targeting proteins with radionuclide. This patent provides for a process for conjugation of a targeting protein with radiolabeled ligand. The process includes several steps: attaching the unradiolabeled ligand to an insoluble support, reaction of the ligand with radionuclide, and conjugation of the radiolabeled ligand with a targeting protein. The targeting protein is selected from the group consisting of antibodies, antibody fragments, monoclonal antibodies, monoclonal antibody fragments, serum proteins, fibrinolytic enzymes, peptide hormones, biologic response modifiers, erythropoietin, and mixtures thereof.

Hansen et al. (U.S. Pat. No. 5,328,679) describe method for radiolabeling a protein with a radioisotope of technetium or rhenium. The protein including antibody or antibody fragment contains a plurality of adjacent free sulfhydryl groups.

Dean (U.S. Pat. No. 5,552,525) describes technetium-99m (Tc-99m) labeled peptides that specifically bind to inflammatory sites in vivo. Goodbody et al. (U.S. Pat. No. 5,866,544) describe peptide-chelator conjugates labeled with radionuclide metal ions, such as technetium-99 m, useful for diagnostic imaging of sites of inflammation.

Konishi et al. (U.S. Pat. No. 6,066,310) relate to a method for tumor diagnosis with a conjugate which consists of radiolabeled avidin. The radiolabeled avidin directly binds to a lectin present on the surface of tumor and the tumor could be detected due to the avidin bound to it.

Magneson et al. (WO 2010/087959 A1) describe composition for radiolabeling DTPA-dextran with Tc-99m.

### SUMMARY OF THE INVENTION

The present invention provides for scintigraphic imaging agents that are compositions comprising radioactively labeled nanoparticles. The compositions of the invention target tumor cells, selectively internalize and accumulate in them, therefore are suitable for early diagnosis of tumors.

In some embodiments, the present invention provides for targeting, self-assembled nanoparticles, which are radiolabeled with Tc-99m. The nanoparticles comprise (i) at least one self-assembled polyelectrolyte biopolymer, (ii) a targeting agent conjugated to a polyelectrolyte biopolymer, and optionally (iii) complexing agent attached to a polyelectrolyte biopolymer. These nanoparticles are radioactively labeled with Tc-99m to produce radiopharmaceutical imaging agent for tumor detection.

More particularly, the self-assembled nanoparticles comprise at least two biocompatible, biodegradable polyelectrolyte biopolymers, where at least one of the polyelectrolyte biopolymers is a polycation and the other of them is a polyanion biopolymer. The nanoparticles have been constructed by the self-assembly of polyanion and polycation biopolymers based on the ion-ion interactions between their functional groups in aqueous media. The targeting moieties are conjugated to one of the self-assembled polyelectrolytes to realize a targeted delivery of particles as contrast agent.

The self-assembled nanosystems may form nanoparticles, which are stable in aqueous media for several weeks. These nanoparticles could be scintigraphic imaging agent by radiolabeling with Tc-99m. The present invention also relates to the composition and method for formation of targeted, radiolabeled nanodevices.

Also provided are methods for making the radiopharmaceutical imaging agent compositions that include step of formation of targeted self-assembled nanoparticles, and step of radiolabeling of nanoparticles with Tc-99m.

The formation of the self-assembled nanoparticulate composition may be influenced by several conditions, such as the pH and the concentration of the solutions, the ratio of polyelectrolytes, the order of mixing, and the ratio of complexing agents.

In a preferred embodiment, one of the polyelectrolyte biopolymers is a polycation, which is preferably chitosan; and the other of the polyelectrolyte biopolymers is a polyanion, which is preferably poly-gamma-glutamic acid.

In a further embodiment, the molecular weight of the chitosan in the nanoparticles ranges from about 20 kDa to 600 kDa, and the molecular weight of the poly-gamma-glutamic acid in the nanoparticles ranges from about 50 kDa to 2500 kDa. In a preferred embodiment, the degree of deacetylation of chitosan ranges between 40% and 99%.

In a preferred embodiment, the targeting agent is preferably folic acid, LHRH, or RGD.

Preferred complexing agents include, but are not limited to: diethylenetriaminepentaacetic acid (DTPA), 1,4,7,10-tetracyclododecane-N,—N′,N″,N′″-tetraacetic acid (DOTA), ethylene-diaminetetraacetic acid (EDTA), 1,4,7,10-tetraazacyclododecane-N,N′,N″-triacetic acid (DO3A), 1,2-diaminocyclohexane-N,N,N′,N′-tetraacetic acid (CHTA), ethylene glycol-bis(beta-aminoethyl ether)N,N,N′,N′,-tetraacetic acid (EGTA), 1,4,8,11-tetraazacyclotradecane-N,N′,N″,N′″-tetraacetic acid (TETA), 1,4,7-triazacyclononane-N,N′,N″-triacetic acid (NOTA) or their reactive derivatives.

In a further embodiment, the nanoparticles have a mean particle size between about 30 and 500 nm, preferably between about 50 and 400 nm, and most preferably between 70 and 250 nm.

The present invention is directed to radioactively labeled scintigraphic imaging agent comprising self-assembled polyelectrolyte biopolymers, targeting agent, and optionally complexing agent. These self-assembled particles internalize into the targeted tumor cells due to the presence of targeting ligands. Due to the specific localization of these targeted radiopharmaceuticals, the early tumor diagnosis could be facilitated.

Table 1 shows the biodistribution of radiolabeled targeted nanoparticles, as percentages of injected activities from ROI data in vivo, in a tumor induced animal model. The targeted nanoparticles were formed by mixing of PGA-FA and CH-DTPA, and after that radiolabeling with Tc-99m was performed.

| | | | | tumorous | | |
|---|---|---|---|---|---|---|
| | | | | left | right | urinary |
| | total | heart | liver | kidney | kidney | bladder |
| 30 min | 100.00%* | 3.87% | 49.83% | 7.16% | 1.95% | 6.21% |
| after | | | | | | |
| injection | | | | | | |
| 8 hrs after | 71.27% | 1.62% | 35.65% | 8.54% | 1.81% | 2.47% |
| inj. | | | | | | |
| 22 hrs ex | — | — | — | 8.46% | 1.78% | — |
| vivo | | | | | | |
| images | | | | | | |

*total measurable activity at T₀

Table 2 shows the biodistribution of radiolabeled targeted nanoparticles 2 hours post injection, as percentages of injected activities from ROI data in vivo, in spontaneously diseased animal model. The targeted nanoparticles were formed by mixing of PGA-FA and CH-DTPA, and after that radiolabeling with Tc-99m was performed.

| | % of Total Counts |
|---|---|
| Heart | 11.97% |
| Liver | 15.76% |
| Kidneys | 8.85% |
| Urinary Bladder | 1.36% |
| Tumor | 3.11% |

### BRIEF DESCRIPTION OF THE DRAWINGS

FIG. 1a shows the schematic representation of the radiolabeling process of targeted nanoparticles. Folated polyanion and polycation-complexone were mixed to produce targeted, self-assembled nanoparticles, and after that radiolabeling with Tc-99m was performed.

FIG. 1b shows the schematic representation of the radiolabeling process of targeted nanoparticles. Self assembled nanoparticles from polyanion and polycation having targeting moiety were formed, and after that radiolabeling with Tc-99m was produced.

FIG. 2 shows the size distribution of a radiolabeled nanoparticulate imaging agent by intensity, 1 h after labeling. The nanoparticles were constructed by self-assembly of biopolymers at a concentration of 0.3 mg/ml, at given ratios, where the CH-DTPA solution was added into the PGA-FA solution. After the nanoparticle formation, radiolabeling with Tc-99m was performed.

FIG. 3 shows the confocal microscopic image of HeDe cells treated with radiolabeled, folate-targeted self-assembled nanoparticles, in which nanoparticles were constructed by self-assembly of folated poly-gamma-glutamic acid and chitosan-DTPA conjugate biopolymers at a ratio of 2:1 and at a concentration of 0.3 mg/ml, where the CH-DTPA solution was added into the PGA-FA solution, and after that radiolabeling with Tc-99m was performed. The confocal microscopic investigation was performed with the cold nanoparticles.

FIG. 4 shows the SPECT scan images of tumor induced animals 30 minutes and 8 hours after injection of targeted nanoparticles radiolabeled with Tc-99m. The targeted nanoparticles were formed by mixing of PGA-FA and CH-DTPA, and after that radiolabeling with Tc-99m was performed.

FIG. 5 shows the ex vivo images and SPECT scans of kidneys of examined tumor induced animal model, 22 hours after injection of targeted nanoparticles radiolabeled with Tc-99. The targeted nanoparticles were formed by mixing of PGA-FA and CH-DTPA, and after that radiolabeling with Tc-99m was performed. Significant accumulation is visible in the tumorous left kidney.

FIG. 6 shows the SPEC/CT images of tumor induced animal model, 6 hours after injection of targeted nanoparticles radiolabeled with Tc-99m. The targeted nanoparticles were formed by mixing of PGA-FA and CH-DTPA, and after that radiolabeling with Tc-99m was performed.

FIG. 7 shows the sagital (a) and coronal (b) SPECT images of a spontaneously diseased dog model, 2 hours after injection of targeted nanoparticles radiolabeled with Tc-99m. The targeted nanoparticles were formed by mixing of PGA-FA and CH-DTPA, and after that radiolabeling with Tc-99m was performed.

FIG. 8 shows the 3D SPEC/CT image of a spontaneously diseased cat model, 2 hours after injection of targeted nanoparticles radiolabeled with Tc-99m. The targeted nanoparticles were formed by mixing of PGA-FA and CH-DTPA, and after that radiolabeling with Tc-99m was performed.

FIG. 9 shows the transversal, coronal and sagital SPECT/CT images of a spontaneously diseased cat model, 2 hours after injection of targeted nanoparticles radiolabeled with Tc-99m. The targeted nanoparticles were formed by mixing of PGA-FA and CH-DTPA, and after that radiolabeling with Tc-99m was performed.

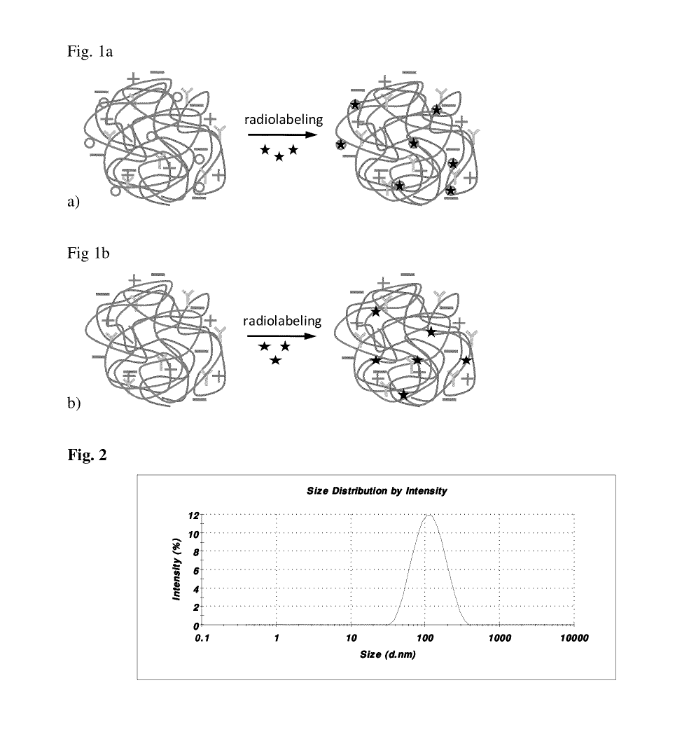

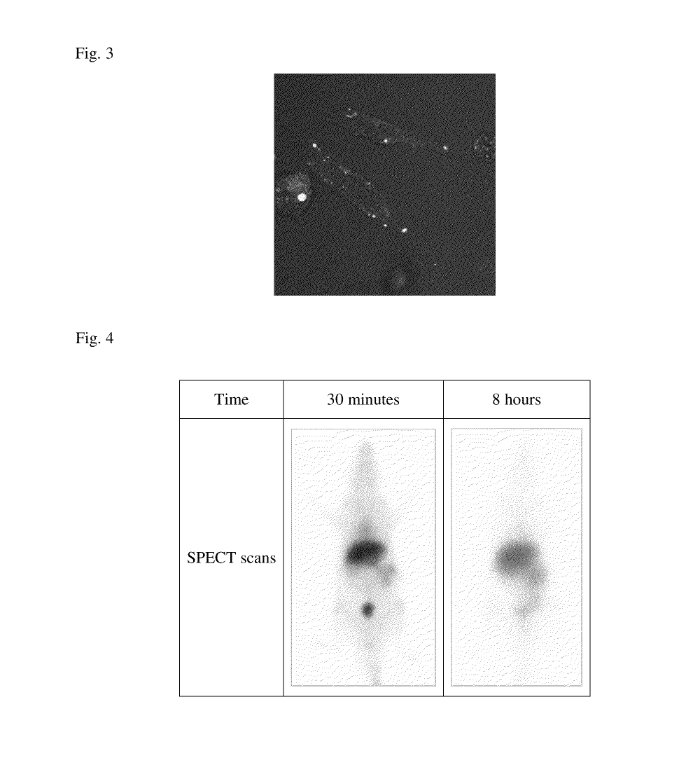

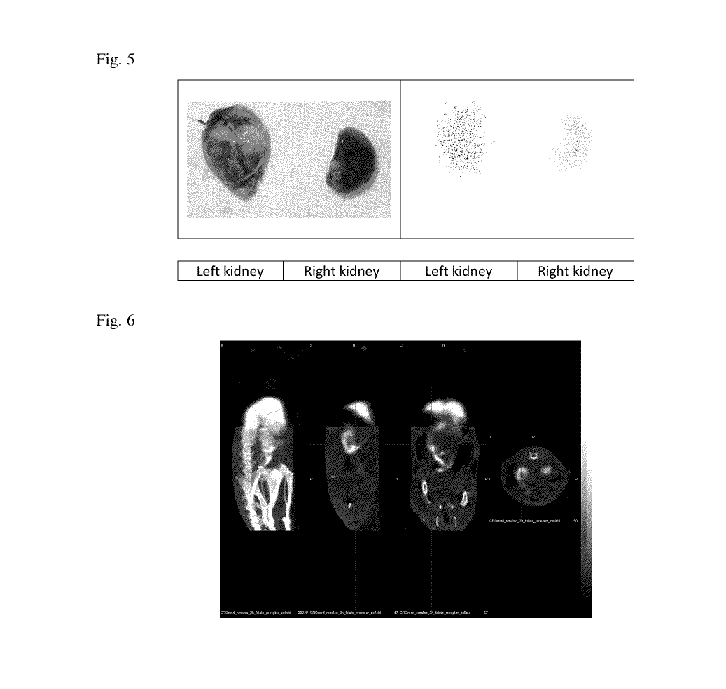

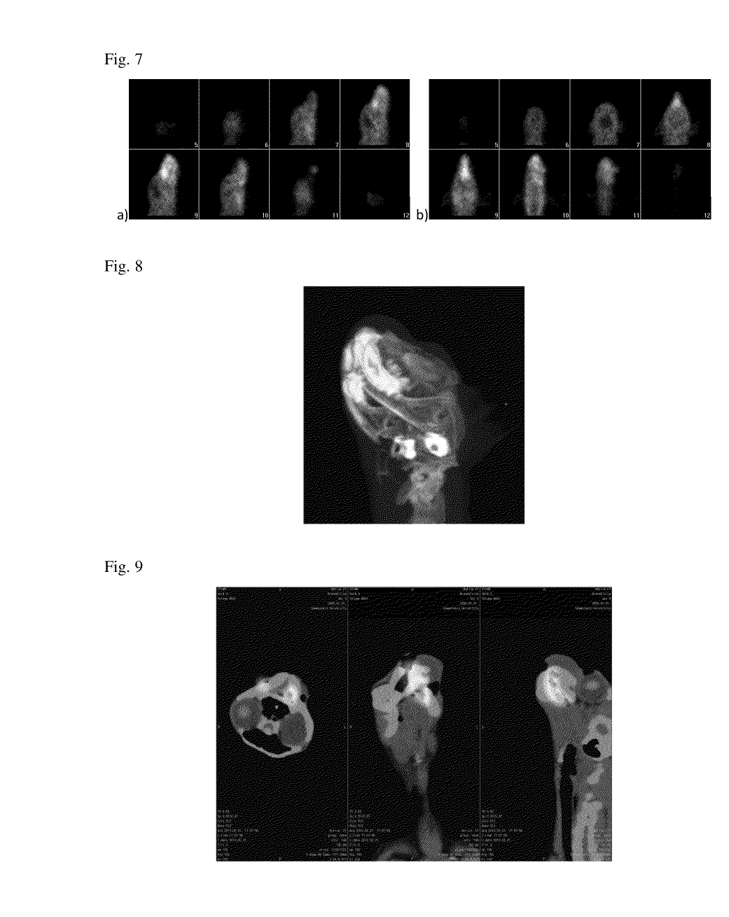

### DETAILED DESCRIPTION OF THE INVENTION

The present invention provides for novel, targeting, self-assembled nanoparticles radiolabeled with Tc-99m as radiodiagnostic composition, methods of using these compositions and methods for preparing such radiolabeled compositions. In preferred embodiments, the nanoparticles are composed of self-assembled polyelectrolyte biopolymers having targeting moieties, which are suitable for targeted delivery of radionuclide metal ions complexed to the nanoparticles. These radiolabeled nanoparticles can specifically bind and internalize into the targeted tumor cells to realize the receptor mediated uptake. Radiolabeled, targeted nanoparticulate composition, methods for making, radiolabeling and using such compositions in the field of diagnosis and therapy are also provided.

### Targeted, Self-Assembled Radiodiagnostic Agent

The present invention is directed to targeted, self-assembled nanoparticles radiolabeled with Tc-99m as potential scintigraphic imaging agent.

In a preferred embodiment, the biocompatible, biodegradable, polymeric nanoparticles are formed by self-assembly via ion-ion interaction of oppositely charged functional groups of polyelectrolyte biopolymers. The nanoparticles contain targeting moieties necessary for targeted delivery of nanosystems.

In a preferred embodiment, the biopolymers are water-soluble, biocompatible, biodegradable polyelectrolyte biopolymers. One of the polyelectrolyte biopolymers is a polycation, positively charged polymers, which is preferably chitosan or its derivatives. The other of the polyelectrolyte biopolymers is a polyanion, a negatively charged biopolymer. The polyanion is preferably selected from a group consisting of polyacrylic acid (PAA), poly-gamma-glutamic acid (PGA), hyaluronic acid (HA), and alginic acid (ALG).

In a preferred embodiment, the molecular weight of the polycation in the nanoparticles ranges from about 20 kDa to 600 kDa, and the molecular weight of the polyanion in the nanoparticles ranges from about 50 kDa to 2500 kDa. In a preferred embodiment, the degree of deacetylation of chitosan ranges between 40% and 99%.

In a preferred embodiment, the targeting agent is coupled covalently to one of the biopolymers using carbodiimide technique in aqueous media. Water soluble carbodiimide, as coupling agent forms amide bonds between the carboxyl and amino functional groups, therefore the targeting ligand could be covalently bound to one of the polyelectrolyte biopolymers.

In the present invention, the preferred targeting agent is selected from folic acid, lutenizing hormone-releasing hormone (LHRH), and an Arg-Gly-Asp (RGD)-containing homodetic cyclic pentapeptide such as cyclo(-RGDf(NMe)V) and the like.

In a preferred embodiment, the most preferred targeting agent is folic acid, which facilitates the folate mediated uptake of nanoparticles, as tumor specific contrast agents. The nanoparticles of the present invention are preferably targeted to tumor and cancer cells, which overexpress folate receptors on their surface. Due to the binding activity of folic acid ligands, the nanoparticles selectively link to the folate receptors held on the surface of targeted tumor cells, internalize and accumulate in the tumor cells.

In a preferred embodiment, the self-assembled nanoparticles comprise a polyanion biopolymer, a polycation biopolymer, a targeting agent covalently attached to one of the biopolymers. In a further embodiment, the self-assembled nanoparticles comprise a polyanion biopolymer, a polycation biopolymer, a targeting agent covalently attached to one of the biopolymers and a complexing agent covalently coupled to the polycation.

In a preferred embodiment, the complexing agent is coupled covalently to the polycation biopolymer. Water-soluble carbodiimide, as coupling agent can be used to make stable amide bonds between the carboxyl and amino functional groups in an aqueous media. Using reactive derivatives of the complexing agents (e.g. succinimide, thiocyanete), the polycation-complexone conjugate can be directly formed in one-step process without any coupling agents.

In a preferred embodiment, the complexing agents are selected form the group of diethylenetriaminepentaacetic acid (DTPA), 1,4,7,10-tetracyclododecane-N,—N′,N″,N′″-tetraacetic acid (DOTA), ethylene-diaminetetraacetic acid (EDTA), 1,4,7,10-tetraazacyclododecane-N,N′,N″-triacetic acid (DO3A), 1,2-diaminocyclohexane-N,N,N′,N′-tetraacetic acid (CHTA), ethylene glycol-bis(beta-aminoethyl ether)N,N,N′,N′,-tetraacetic acid (EGTA), 1,4,8,11-tetraazacyclotradecane-N,N′,N″,N′″-tetraacetic acid (TETA), 1,4,7-triazacyclononane-N,N′,N″-triacetic acid (NOTA) or their reactive derivatives. More preferably, the complexing agents are DOTA, DTPA, EDTA and DO3A, most preferably DTPA.

In a preferred embodiment, the nanoparticles described herein have a hydrodynamic diameter between about 30 and 500 nm, preferably between about 50 and 400 nm, and the most preferred range of the hydrodynamic size of nanoparticles is between 70 and 250 nm.

In a preferred embodiment, the targeted, self-assembled nanoparticles described herein are radiolabeled with radionuclide metal ion, which is preferably Tc-99m.

In a preferred embodiment, the radionuclide metal ions are homogeneously distributed throughout the self-assembled nanoparticle. The radionuclide metal ions can make stable complexes with the residual carboxyl functional groups of the polyanion, which is self-assembling into the nanoparticles. In further embodiment, the radionuclide metal ions can make stable complex with the free complexing agents attached to the polycation biopolymer, therefore they could be performed homogeneously dispersed.

In a preferred embodiment, the complexing agent is coupled covalently to the polycation biopolymer. The nanoparticles make stable complex with the radioactive metal ions through these complexone ligands.

### Methods of Making Targeted, Self-Assembled Radiodiagnostic Compositions

The present invention is directed to novel, radiolabeled targeting nanoparticles as scintigraphic imaging agent. The nanoparticle compositions described herein are prepared by self-assembly of oppositely charged polyelectrolytes via ion-ion interaction between their functional groups. The targeting ligands are conjugated covalently to one of the polyelectrolyte biopolymers and optionally complexing agents covalently conjugated to the polycation biopolymer. These targeted nanoparticles are radioactively labeled with Tc-99m to produce radiodiagnostic agent.

In a preferred embodiment, the targeting ligands of the nanoparticles are attached to one of the biopolymers covalently. The targeting agent is preferably folic acid, LHRH, RGD, the most preferably folic acid.

A polyanion via its reactive carboxyl functional groups and a polycation via its reactive amino functional groups can form stable amide bond with the functional groups of folic acid using carbodiimide technique. In the present invention, folated biopolymers meaning folated polyanion or folated polycation can be used for the formation of nanoparticles, as targeted nanoparticulate systems.

In a preferred embodiment, the polycation or its derivatives are used for the formation of nanoparticles. In the preferred embodiment a polycation without any covalent modification is used for the formation of self-assembled nanoparticles. In further embodiment, derivatives of the polycation are produced by coupling the complexing agent to it covalently. In the present invention several complexing agents having reactive carboxyl groups are used to make stable complex with metal ions and therefore afford possibility to use these systems as imaging agent.

In a preferred embodiment, four types of polycations can be used for the formation of nanoparticles: (i) a polycation without any covalent modification; (ii) a targeted polycation, when the targeting agent is coupled covalently to the polycation; (iii) a polycation-complexone conjugate, when the complexing agent is covalently attached to the polycation; and (iv) a targeted polycation-complexone conjugate, when targeting moiety and the complexing agent are covalently coupled to the polycation biopolymer.

In a preferred embodiment, for the formation of polycation-complexone conjugation, the concentration of the biopolymer ranges between about 0.05 mg/ml and 5 mg/ml, preferably 0.1 mg/ml and 2 mg/ml, and most preferably 0.3 mg/ml and 1 mg/ml.

In a preferred embodiment, the overall degree of substitution of complexing agent in polycation-complexone conjugate is generally in the range of about 1-50%, preferably in the range of about 5-30%, and most preferably in the range of about 10-20%.

The nanoparticles can be formed independently of order of addition. In a preferred embodiment the polycation or its derivatives and the polyanion or its derivatives are mixed to produce stable nanoparticles.

The nanoparticle compositions described herein are prepared by mixing aqueous solutions of a polyanion or modified polyanion, a polycation or modified polycation at given ratios and orders of addition. In a preferred embodiment, the concentration of biopolymers ranges between about 0.005 mg/ml and 2 mg/ml, preferably between 0.2 mg/ml and 1 mg/ml, most preferably 0.3 mg/ml and 0.5 mg/ml. The concentration ratio of biopolymers mixed is about 2:1 to 1:2, most preferably about 1:1. The biopolymers are mixed in a weight ratio of 6:1 to 1:6, most preferably 3:1 to 1:3.

The pH of the biopolymer solution is one of the main factors, which influence the nanoparticle formation due to the surface charge of biopolymers. In a preferred embodiment for the nanoparticle formation, the pH of polycation or its derivatives varies between 3.5 and 6.0, and the pH of aqueous solution of polyanion or its derivatives ranges between 7.5 and 9.5.

In a preferred embodiment, biopolymers with high charge density form stable nanoparticles due to the given pH values. The surface charge of nanoparticles could be influenced by several reaction parameters, such as ratio of biopolymers, ratio of residual functional groups of biopolymers, pH of the biopolymers and the environment, etc, the selection of the parameters belongs to the knowledge of the skilled person The electrophoretic mobility values of nanoparticles, showing their surface charge, could be positive or negative, preferably negative, depending on the reaction conditions described above.

In a preferred embodiment, nanoparticulate compositions, as targeted, radiolabeled scintigraphic imaging agents are provided. The radionuclide metal ion is preferably technetium-99m. The preferred radioactive metal ions can make stable complex with the targeting, self-assembled nanoparticles due the residual carboxyl functional groups of polyanion or due to the complexing agents, which are covalently conjugated to polycation.

In a preferred embodiment, the targeted, self-assembled nanoparticles are radiolabeled with Tc-99m to produce radiodiagnostic imaging agents. The radiolabeling takes place in physiological salt solution. For labeling, SnCl₂ (×2H₂O) as reducing agent is added to nanoparticles, then generator-eluted sodium pertechnetate (^{99m}TcO₄⁻) is added to the solvent. The incubation temperature for radiolabeling is room temperature, the incubation time for radiolabeling ranges preferably between 2 min and 120 min, more preferably 5 min and 90 min, and the most preferably 30 min and 60 min

### Methods of Using Targeted, Self-Assembled Radiodiagnostic Compositions

The radiolabeled nanoparticle compositions, as radiodiagnostic agents are useful for the targeted delivery of radionuclide metal ions complexed to the nanoparticles. The present invention is directed to methods of using the above-described nanoparticles, as targeted, radiopharmaceutical imaging agent.

In a preferred embodiment, the nanoparticles as nanocarriers deliver the radionuclide metal ions to the targeted tumor cells in vitro, therefore can be used as targeted, radioactively labeled scintigraphic imaging agents. The radiolabeled nanoparticles internalize and accumulate in the targeted tumor cells, which overexpress folate receptors, to facilitate the early tumor diagnosis. The side effect of these contrast agents is minimal, because of the receptor mediated uptake of nanoparticles.

In a preferred embodiment, the radioactively labeled, targeted imaging agents are stable at pH 7.4, they may be injected intravenously. Based on the blood circulation, the nanoparticles could be transported to the area of interest.

The ability of the radiopharmaceutical nanoparticles to be internalized was studied in cultured cancer cells, which overexpresses folate receptors using confocal microscopy. Due to the folic acid, as targeting moiety, the nanoparticles efficiently internalize into the targeted tumor cells, which overexpress folate receptors.

Specific localization, accumulation and biodistribution of these radioactively labeled targeted nanoparticles were investigated in vivo using tumor induced rat animal model and spontaneously diseased dog animal model. Targeted, radiolabeled nanoparticles specifically internalize into the tumor cells overexpressing folate receptors on their surface. The specific localization was examined by SPECT method, and the biodistribution was estimated by quantitative ROI analysis.

Whole-body biodistribution of the nanoparticles revealed significant higher uptake in the tumorous kidney for a long time compared to the non-tumorous contralateral side. Lungs- and thyroids uptake was under detectable range, which confirm the stability of nanoparticles in vivo.

In vivo SPECT and SPECT/CT imaging of tumor-induced rats and spontaneous diseased dog and cat animal models reinforce visually the uptake results, which are in accordance with the biodistribution data. Significant and durable higher left kidney uptake was observed for the radiolabeled targeted nanoparticles in tumour induced Wistar rats in the spontaneously diseased dog and cat animals compared to contralateral side intensity.

The new nanoparticles as targeted contrast agent improve the tumour targeting and are able to detect folate-receptor overexpressing tumours in animal models with enhanced contrast.

The use of targeted, radiolabeled nanoparticles, as scintigraphic imaging agent enhances the receptor mediated uptake, therefore these nanoparticles can be attractive candidates as nanocarriers for radionuclide metal ions.

The rapid, simple and reproducible labeling and radiochemical stability of nanoparticles with in vivo size stability and non-toxicity is allowed the possibility of a relative rapid manufacture and clinical use of the new product.

### EXAMPLES

### Example 1

### Preparation of Folated poly-gamma-glutamic acid (γ-PGA)

Folic acid was conjugated via the amino groups to γ-PGA using carbodiimide technique. γ-PGA (m=60 mg) was dissolved in water (V=100 ml) to produce aqueous solution. The pH of the polymer solution was adjusted to 6.0. After the dropwise addition of cold water-soluble 1-[3-(dimethylamino)propyl]-3-ethylcarbodiimide methiodide (CDI) (m=13 mg in 2 ml distilled water) to the γ-PGA aqueous solution, the reaction mixture was stirred at 4° C. for 1 h, then at room temperature for 1 h. After that, folic acid (m=22 mg in dimethyl sulfoxide, V=10 ml) was added dropwise to the reaction mixture and stirred 4° C. for 1 h, then at room temperature for 24 h. The folated poly-γ-glutamic acid (γ-PGA-FA) was purified by dialysis.

### Example 2

### Preparation of Folated Chitosan

A solution of 1-(3-dimethylaminopropyl)-3-ethylcarbodiimide hydrochloride (CDI) and FA in anhydrous DMSO was prepared and stirred at room temperature until FA was well dissolved (1 h). Chitosan was dissolved in 0.1 M hydrochloric acid, to produce a solution with a concentration of 1 mg/ml, and then adjusted to pH 5.5 with 0.10 M sodium hydroxide solution. After the dropwise addition of CDI (m=5.1 mg in 1 ml distilled water) to the chitosan solution (V=20 ml), the reaction mixture was stirred for 10 min. Then folic acid (m=8.5 mg in dimethyl sulfoxide, V=1 ml) was added to the reaction mixture. The resulting mixture was stirred at room temperature in the dark for 24 h. It was brought to pH 9.0 by dropwise addition of diluted aqueous NaOH and was washed three times with aqueous NaOH, and once with distilled water. The polymer was isolated by lyophilization.

### Example 3

### Preparation of Chitosan-DTPA Conjugate

Chitosan (m=15 mg) was solubilized in water (V=15 ml); its dissolution was facilitated by dropwise addition of 0.1 M HCl solution. After the dissolution, the pH of chitosan solution was adjusted to 5.0. After the dropwise addition of DTPA aqueous solution (m=11 mg, V=2 ml, pH=3.2), the reaction mixture was stirred at room temperature for 30 min, and at 4° C. for 15 min. after that, CDI (m=8 mg, V=2 ml distilled water) was added dropwise to the reaction mixture and stirred at 4° C. for 4 h, then at room temperature for 20 h. The chitosan-DTPA conjugate (CH-DTPA) was purified by dialysis.

### Example 4

### Preparation of Chitosan-DOTA Conjugate

Chitosan (m=5 mg) was solubilized in water (V=5 ml); its dissolution was facilitated by dropwise addition of 0.1M HCl solution. After the dissolution, the pH of chitosan solution was adjusted to 7.0. 2,2′,2″-(10-(1-carboxy-4-((4-isothiocyanatobenzyl)amino)-4-oxobutyl)-1,4,7,10-tetraazacyclododecane-1,4,7-triyl)triacetic acid was dissolved in DMSO (m=5.6 mg, V=1 ml) to produce a solution. After the dropwise addition of DOTA solution the reaction mixture was stirred at room temperature for 2 h. The chitosan-DOTA conjugate (CH-DOTA) was purified by dialysis.

### Example 5

### Preparation of Self-Assembled Nanoparticles

Stable self-assembled nanoparticles were developed via an ionotropic gelation process between the folated poly-γ-glutamic acid (γ-PGA-FA), and chitosan-DTPA conjugate (CH-DTPA). Briefly, CH-DTPA solution (c=0.3 mg/ml, V=1 ml, pH=4.0) was added into γ-PGA-FA solution (c=0.3 mg/ml, V=2 ml, pH=9.5) under continuous stirring at room temperature for 30 min. An opaque aqueous colloidal system was gained, which remained stable at room temperature for several weeks at physiological pH. (γ-PGA-FA/CH-DTPA)

### Example 5

### Preparation of Self-Assembled Nanoparticles

Stable self-assembled nanoparticles were developed via an ionotropic gelation process between the folated poly-γ-glutamic acid (γ-PGA-FA), chitosan-DOTA conjugate (CH-DOTA). Briefly, CH-DOTA solution (c=0.1 mg/ml, V=1 ml, pH=5.0) was added into γ-PGA-FA solution (c=0.1 mg/ml, V=1 ml, pH=8.0) under continuous stirring at room temperature for 30 min. An opaque aqueous colloidal system was gained, which remained stable at room temperature for several weeks at physiological pH. (γ-PGA-FA/CH-DOTA)

### Example 6

### Characterization of Self-Assembled Nanoparticles

The hydrodynamic size and size distribution of particles was measured using a dynamic light scattering (DLS) technique with a Zetasizer Nano ZS (Malvern Instruments Ltd., Grovewood, Worcestershire, UK). This system is equipped with a 4 mW helium/neon laser with a wavelength of 633 nm and measures the particle size with the noninvasive backscattering technology at a detection angle of 173°. Particle size measurements were performed using a particle-sizing cell in the automatic mode. The mean hydrodynamic diameter was calculated from the autocorrelation function of the intensity of light scattered from the particles.

### Example 7

### Cellular Uptake of Targeted Nanoparticles

Internalization and selectivity of targeted nanoparticles was investigated in Hepatocellular carcinoma (HeDe) cancer cells, which overexpress folate receptors by using confocal microscopy. The samples were imaged on an Olympus FluoView 1000 confocal microscope. Excitation was performed by using the 488 nm line of an Ar ion laser (detection: 500-550 nm) to image Alexa 488. Images were analyzed using Olympus FV10-ASW 1.5 software package.

The nanoparticles internalized and accumulated in the targeted tumor cells. Folic acid, as targeting agent is specific to cancer cells, which overexpress folate receptors. Due to this targeting moiety, enhanced receptor mediated cellular uptake of the novel self-assembled nanoparticles can be observed. Therefore these nanoparticles can be attractive candidates as tumor specific nanocarriers.

### Example 8

### Radiolabeling Method and In Vitro Radiochemical Stability of Nanoparticles

For labeling, 40 μg SnCl₂ (×2H₂O) (in 10 μl 0.1M HCl) as reducing agent was added to 2.6 ml of ligand, then 1 ml (900 MBq activity) of generator-eluted sodium pertechnetate (^{99m}TcO₄⁻) was added to the solvent. Labeling was performed in 60 min incubation at room temperature.

Radiochemical purity was examined by means of thin layer chromatography, using silica gel as the coating substance on a glass-fibre sheet (ITLC-SG). Plates were developed in methyl ethyl ketone. Raytest MiniGita device was applied (Mini Gamma Isotope Thin Layer Analyzer) in Radiopharmacy Ltd. to determine the distribution of radioactivity in developed ITLC-SG plates. Labeling efficiency was examined 1 h, 6 h and 24 h after labeling. Radiochemical samples were stored at room temperature in dark place.

### Example 9

### Biodistribution of Radiolabeled, Targeted Nanoparticles in Animal Models

Solution containing radiolabeled, targeted nanoparticulate compound (V=0.5 mL, 125 MBq of ^{99m}Tc) as obtained in Example 7 was administered through the tail vein of the tumor induced rat animal model. Critical organs eg.: heart, liver, kidneys and urinary bladder was drawn around, and organ uptakes were estimated by quantitative ROI analysis. Results are reported in Table 1.

Solution containing radiolabeled, targeted nanoparticulate compound (V=2.0 mL, 500 MBq of ^{99m}Tc) as obtained in Example 7 was administered through the cephalic vein of the spontaneously diseased dog animal model. Critical organs eg.: heart, liver, kidneys and urinary bladder was drawn around, and organ uptakes were estimated by quantitative ROI analysis. Results are reported in Table 2.

### Example 10

### SPECT Images of Animal Models after Injection of Radiolabeled, Targeted Nanoparticles

Dorso-ventral and left-lateral images were taken with a single-head digital SPECT gamma camera (Nucline X-ring, Mediso) at 30 min and 8 hours post-injection using a LEHR collimator to determine the in vivo localization of injected radioactivity. 22 hours after injection ex vivo images were taken with both kidneys of two experimental animals. Prior to the imaging the animals were anaesthetized by administering a combination of xylazine hydrochloride and ketamine hydrochloride intraperitoneal. The gamma camera was previously calibrated for the 140 keV gamma photon of 99 mTc. All the images were acquired with 60 seconds time-prerequisits using a 1024×1024×16 matrix size.

Moreover, 2 hours after injection whole-body fusion images were taken by a laboratory SPECT/CT hybrid scanner camera (nanoSPECT/CT, Mediso Ltd, Hungary) for more detailed images.

1. A scintigraphic imaging composition suitable for targeting tumor cells, and selectively internalizing and accumulating in them, said composition comprising self-assembled nanoparticles, and radionuclide metal ions, preferably Tc-99m complexed to the nanoparticles, wherein the nanoparticles comprise (i) at least one self-assembled, preferably water-soluble polyelectrolyte biopolymer, (ii) a targeting agent conjugated to a polyelectrolyte biopolymer, and optionally (iii) a complexing agent attached to a polyelectrolyte biopolymer.

2. The scintigraphic imaging composition according to claim 1, wherein the self-assembled nanoparticles comprise at least two biocompatible, biodegradable polyelectrolyte biopolymers, wherein at least one of the polyelectrolyte biopolymers is a polycation or a derivative thereof and the other of them is a polyanion or a derivative thereof.

3. The scintigraphic imaging composition according to claim 1, wherein the self-assembled nanoparticles are constructed by self-assembly of polyanion and polycation biopolymers based on the ion-ion interactions between their functional groups, preferably in an aqueous media.

4. The scintigraphic imaging composition according to claim 1, wherein
a) one of the polyelectrolyte biopolymers is a polycation, which is preferably chitosan, preferably having a molecular weight from about 20 kDa to 600 kDa, preferably the degree of deacetylation of chitosan ranges between 40% and 99%, said polycation optionally (i) being without any covalent modification; (ii) having the targeting agent coupled covalently to the polycation; (iii) being in the form of a polycation-complexone conjugate, when the complexing agent is covalently attached to the polycation; or (iv) being in the form of a polycation-complexone conjugate, where the targeting moiety and the complexing agent are covalently coupled to the polycation; and/or
b) the other of the polyelectrolyte biopolymers is a polyanion, preferably selected from the group consisting of polyacrylic acid (PAA), poly-gamma-glutamic acid (PGA), hyaluronic acid (HA), and alginic acid (ALG), preferably poly-gamma-glutamic acid (PGA), preferably having a molecular weight from about 50 kDa to 2500 kDa; and/or
c) the targeting agent is preferably covalently attached to one of the biopolymers preferably in an aqueous media, and preferably is selected from the group of folic acid, LHRH, RGD, most preferably folic acid; and/or
d) the complexing agent is preferably covalently coupled to the polycation, and is preferably selected from the group consisting of diethylenetriaminepentaacetic acid (DTPA), 1,4,7,10-tetracyclododecane-N,—N′,N″,N′″-tetraacetic acid (DOTA), ethylene-diaminetetraacetic acid (EDTA), 1,4,7,10-tetraazacyclododecane-N,N′,N″-triacetic acid (DO3A), 1,2-diaminocyclohexane-N,N,N′,N′-tetraacetic acid (CHTA), ethylene glycol-bis(beta-aminoethyl ether)N,N,N′,N′,-tetraacetic acid (EGTA), 1,4,8,11-tetraazacyclotradecane-N,N′,N″,N″-tetraacetic acid (TETA), 1,4,7-triazacyclononane-N,N′,N″-triacetic acid (NOTA) and their reactive derivatives; more preferably, the complexing agents are DOTA, DTPA, EDTA and DO3A, most preferably DTPA; and/or
e) the radionuclide metal ions are homogeneously distributed throughout the self-assembled nanoparticle;

5. The scintigraphic imaging composition according to claim 1, wherein the nanoparticles have a mean particle size between about 30 and 500 nm, preferably between about 50 and 400 nm, and most preferably between 70 and 250 nm hydrodynamic diameter.

6. A process for the preparation of the scintigraphic imaging composition according to claim 1 comprising the steps of
a) forming of targeted self-assembled nanoparticles by the self-assembly of oppositely charged polyelectrolytes preferably by mixing the polycation or its derivative and the polyanion or its derivative to produce stable nanoparticles; and
b) radiolabeling of the nanoparticles with radionuclide metal ions, preferably Tc-99m.

7. The process according to claim 6, wherein a polycation without any covalent modification is used for the formation of self-assembled nanoparticles.

8. The process according to claim 6, wherein the polycation used is produced by coupling a complexing agent, preferably a complexing agent having reactive carboxyl groups to said polycation covalently

9. The process according to claim 6, wherein the concentration of the biopolymer used ranges between about 0.05 mg/ml and 5 mg/ml, preferably 0.1 mg/ml and 2 mg/ml, and most preferably 0.3 mg/ml and 1 mg/ml.

10. The process according to claim 6, wherein the overall degree of substitution of the complexing agent used is in the range of about 1 to 50%, preferably in the range of about 5 to 30%, and most preferably in the range of about 10 to 20%.

11. The process according to claim 6, wherein aqueous solutions of the polyanion or modified polyanion, and polycation or modified polycation is mixed preferably a concentration between about 0.005 mg/ml and 2 mg/ml, preferably between 0.2 mg/ml and 1 mg/ml, most preferably 0.3 mg/ml and 0.5 mg/ml.

12. The process according to claim 6, wherein the concentration ratio of the biopolymers mixed is about 2:1 to 1:2, most preferably about 1:1.

13. The process according to claim 6, wherein the weight ratio of the biopolymers mixed is 6:1 to 1:6, most preferably 3:1 to 1:3.

14. The process according to claim 6, wherein the pH of polycation or its derivative used ranges between 3.5 and 6.0, and the pH of aqueous solution of the polyanion or its derivative used ranges between 7.5 and 9.5.

15. The process according to claim 6, wherein the radiolabeling of nanoparticles with radionuclide metal ions, preferably Tc-99m is performed in physiological salt solution, by adding SnCl₂ (×2H₂O) as reducing agent to the nanoparticles, then adding sodium pertechnetate (^{99m}TcO₄⁻) to the solvent, at room temperature, for an incubation time between 2 min and 120 min, more preferably 5 min and 90 min, and the most preferably 30 min and 60 min.

16. A method for targeted radiopharmaceutical imaging, said method comprising administering the scintigraphic imaging composition of claim 1 to a subject.

17. The method according to claim 16, wherein the radioactively labeled, targeted imaging agents are injected intravenously.
